(19) United States
(12) Patent Application Publication
(10) Pub. No.: US 2004/0242658 A1
(43) Pub. Date: Dec. 2, 2004

(54) Amorphous form of rosiglitazone maleate and process for preparation thereof

(75) Inventors: Manne Satyanarayana Reddy, Hyderabad (IN); Gudipati Srinivasulu, Hyderabad (IN); Mandava Venkata Naga Brahmeshwar Rao, Hyderabad (IN); Banda Goverdhan Reddy, Hyderabad (IN)

Correspondence Address: Janet I. Cord, Ladas & Parry, 26 West 61 Street, New York, NY, 10023, US

(73) Assignees: DR. REDDY'S LABORATORIES LIMITED; DR. REDDY'S LABORATORIES, INC.

(21) Appl. No.: 10/754,096

(22) Filed: Jan. 7, 2004

(30) Foreign Application Priority Data
Jan. 8, 2003 (IN) 12/MAS/2003

(51) Int. Cl. A61K 31/426; C07D 277/16

(52) U.S. Cl. 514/369; 548/182

(57) ABSTRACT

An amorphous rosiglitazone maleate and its preparation as well as uses thereof for a pharmaceutical composition and a method for medical treatment including combination therapy are described.

### CROSS REFERENCE TO RELAYTED APPLICATION

This application claims priority of Indian Patent Application No. 12/MAS/2003, filed on Jan. 8, 2003, the disclosure of which is incorporated herein by reference in its entirety.

### BACKGROUND OF THE INVENTION

Rosiglitazone, 5-[4-[2-(N-methyl-N-(2-pyridyl)amino)ethoxy]-benzyl]-2,4-thiazolidinedione maleate is a highly selective and potent agonist for the peroxisome proliferators-activated receptor-gamma (PPARγ). Rosiglitazone maleate is used for the management of type 2 diabetes mellitus, also called non-insulin-dependent diabetes mellitus (NIDDM). Rosiglitazone maleate is believed to act primarily by increasing insulin sensitivity and improving glycemic control while reducing circulating insulin levels.

### SUMMARY OF THE INVENTION

It has been reported previously that amorphous forms of certain drugs exhibit distinct dissolution characteristics and in some cases distinct bioavailability patterns compared to the crystalline form. (See, e.g., Konno T, (1990) Chem. Pharm. Bull. 38:2003-2007). The dissolution rate may favor one formulation over another. For some therapeutic indications, one formulation may be favored over another. Similarly one formulation may be more suitable for treating certain patient populations. Therefore, it is desirable to have a procedure for making amorphous product or for converting a crystalline form of the drug to the amorphous form.

The invention relates to an amorphous form of rosiglitazone maleate. Preferably, the amorphous form of rosiglitazone maleate may have substantially the same X-ray diffraction pattern as shown in FIG. 1. Various embodiments and variants are provided.

The invention also relates to a composition that includes rosiglitazone maleate in a solid form, wherein at least 80% by weight of the solid rosiglitazone maleate is in an amorphous form.

The invention also relates to a process for preparation of an amorphous form of rosiglitazone maleate.

The invention also relates to a pharmaceutical composition that includes an amorphous form of rosiglitazone maleate and one or more pharmaceutically acceptable carriers or diluents. The pharmaceutical composition include comprise one or more additional active ingredients in addition to rosiglitazone maleate. Preferably, the pharmaceutical composition is in a solid dosage form for oral administration, such as a tablet.

The invention also relates to a method for treatment or prophylaxis of diabetes mellitus, conditions associated with diabetes mellitus and complication thereof in a mammal.

### BRIEF DESCRIPTION OF THE ACCOMPANYING DRAWINGS

FIG. 1 shows a sample X-ray power diffractogram of an amorphous form of rosiglitazone maleate.

### DESCRIPTION OF THE INVENTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

Unless stated to the contrary, any use of the words such as “including,” “containing,” “comprising,” “having” and the like, means “including without limitation” and shall not be construed to limit any general statement that it follows to the specific or similar items or matters immediately following it. Embodiments of the invention are not mutually exclusive, but may be implemented in various combinations. The described embodiments of the invention and the disclosed examples are given for the purpose of illustration rather than limitation of the invention as set forth the appended claims.

For purposes of the present invention, the following terms are defined below. “Pharmaceutically acceptable” means that which is useful in preparing a pharmaceutical composition that is generally non-toxic and is not biologically undesirable and includes that which is acceptable for veterinary use and/or human pharmaceutical use.

The term “composition” includes, but is not limited to, a powder, a suspension, an emulsion and/or mixtures thereof. The term composition is intended to encompass a product containing the specified ingredients in the specified amounts, as well as any product, which results, directly or indirectly, from combination of the specified ingredients in the specified amounts. A “composition” may contain a single compound or a mixture of compounds. A “compound” is a chemical substance that includes molecules of the same chemical structure regardless of its three dimensional orientation. Thus, it may be used to indicate racemates, stereoisomers, or both.

The term “pharmaceutical composition” is intended to encompass a product including the active ingredient(s), pharmaceutically acceptable excipients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing the active ingredient, additional active ingredient(s), and pharmaceutically acceptable excipients.

The term “excipient” means a component of a pharmaceutical product that is not the active ingredient, such as filler, diluent, carrier, and so on. The excipients that are useful in preparing a pharmaceutical composition are preferably generally safe, non-toxic and neither biologically nor otherwise undesirable, and are acceptable for veterinary use as well as human pharmaceutical use. “A pharmaceutically acceptable excipient” as used in the specification and claims includes both one and more than one such excipient.

When used herein, the expression “prophylaxis of conditions associated with diabetes mellitus” includes the treatment of conditions such as insulin resistance, impaired glucose tolerance, hyperinsulinaemia and gestational diabetes.

The term “isolating” is used to indicate separation of the compound being isolated regardless of the purity of the isolated compound from any unwanted substance, which is present with the compound as a mixture.

The term “substantially free of” in reference to a composition, as used herein, means that the absent substance cannot be detected in the composition by methods known to those skilled in the art at the time of the filing of this application.

Rosiglitazone maleate has the chemical structure:

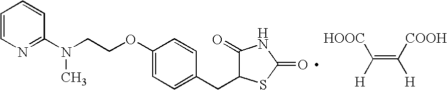

Rosiglitazone maleate compound itself may be prepared according to known procedures such as those disclosed in U.S. Pat. Nos. 5,002,953; 5,646,169; 5,741,803; and 6,288,095 of which the disclosures are incorporated herein by reference.

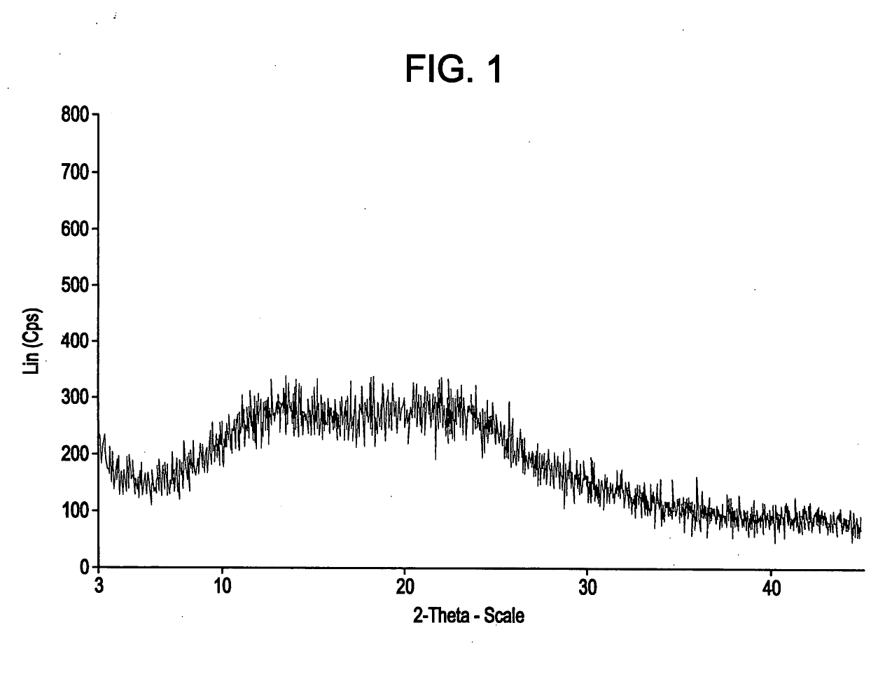

According to one aspect of the invention, there is provided rosiglitazone in an amorphous form. Amorphous materials do not exhibit the three-dimensional long-range order found in crystalline materials but are structurally more similar to liquids where the arrangement of molecules is random. Amorphous solids are not crystalline and therefore do not give a definitive x-ray diffraction pattern (XRD), in addition they do not give rise to a melting point and tend to liquefy at some point beyond the glass transition point (Hancock and Zografi, (1997) J. Pharm. Sci., 86:1-12). A sample of an XRD spectra of rosaglidazone maleate obtained by the inventors is shown in FIG. 1. As seen therefrom, the XRD pattern is highly characteristic of an amorphous solid. The X-ray diffractogram was measured on Bruker Axe, DS advance Power X-ray Diffractometer with Cu K alpha-1 Radiation source. A particular process for preparation of the amorphous form of rosiglitazone maleate is also provided and includes: a) providing rosiglitazone maleate solution in a nitrile solvent, an alcoholic solvent or a mixture thereof; b) removing the solvent to form a solid residue; and c) isolating the solid residue to obtain the amorphous form of rosiglitazone maleate. The preferred nitrile solvents include this acetonitrile, and propionitrile. The preferred alcoholic solvents include methanol, ethanol, isopropyalcohol, n-butyl alcohol, t-butyl alcohol and mixtures thereof.

The removal of the solvent from the rosiglitazone maleate solution may be affected at an increased temperature, preferably at reflux temperature, and/or reduced pressure. Preferably, the removal of the solvent may be carried out preferably below 75° C., more preferably at about 40° C.-75° C., yet more preferably at about 60° C.-75° C., yet more preferably at about 70° C.-75° C. The solid residue obtained after solvent removal may be isolated and dried using conventional methods. The advantages of the process include simplicity, ecofriendliness and suitability for commercial use. The amorphous form of rosiglitazone maleate shown in FIG. 1 is produced by described process.

The invention also relates to a composition of solid rosiglitazone maleate wherein at least 80% of the total weight of rosiglitazone maleate is in the amorphous form. In a preferred form of this composition, the solid rosiglitazone maleate is suitable for use as a bulk active ingredient in formulating pharmaceutical products. The remainder of the solid rosiglitazone maleate in the composition, i.e., 20% or less of the total weight of rosiglitazone maleate, may be other forms of rosiglitazone maleate, e.g. crystalline forms or polymorphs. Several crystalline forms of rosiglitazone maleate are known and are disclosed in WO 00/64896, WO 00/64893, WO 00/64893, WO 99/31093, WO 99/31094, and WO 99/311095, of which entire contents are incorporated herein by reference.

In an embodiment of the invention, the composition may include at least 95% of the amorphous form of rosiglitazone maleate with respect to total weight of the solid rosiglitazone maleate in the composition. In another embodiment of the invention, the composition may include at least 99% of the amorphous form of rosiglitazone maleate with respect to total weight of the solid rosiglitazone maleate in the composition. In yet another embodiment of the invention, the composition is substantially free of any forms of rosiglitazone maleate other than its amorphous form.

The preferred method of differentiating amorphous rosiglitazone maleate from other crystalline and non-crystalline forms of rosiglitazone maleate is X-ray powder diffraction (XPD). The XPD pattern of pure amorphous rosiglitazone maleate, as illustrated in FIG. 1, can be seen to lack discernible acute peaks. Thus, amorphous rosiglitazone maleate, according to the present invention, is characterized in providing an X-ray powder diffraction pattern containing one or more broad diffuse halos having very low counts (i.e. see FIG. 1) in contrast to the sharp diffraction peaks characteristic of crystalline materials. Of course it will be appreciated that a mixture comprising detectable amounts of both crystalline and amorphous rosiglitazone maleate will exhibit both the characteristic sharp peaks and the diffuse halo(s) on XPD. This will be evident by an increase in the baseline and also a reduction in crystalline peak intensities.

X-ray diffraction also provides a convenient and practical means for quantitative determination of the relative amounts of crystalline and/or amorphous forms in a solid mixture. X-ray diffraction is adaptable to quantitative applications because the intensities of the diffraction peaks of a given compound in a mixture are proportional to the fraction of the corresponding powder in the mixture. The percent composition of amorphous or crystalline forms of rosiglitazone maleate in an unknown composition can be determined. Preferably, the measurements are made on solid powder rosiglitazone maleate. The X-ray powder diffraction patterns of an unknown composition can be compared to known quantitative standards containing pure crystalline forms of rosiglitazone maleate to identify the percent ratio of a particular crystalline form. This is done by comparing the relative intensities of the peaks from the diffraction pattern of the unknown solid powder composition with a calibration curve derived from the X-ray diffraction patterns of pure known samples. The curve can be calibrated based on the X-ray powder diffraction pattern for the strongest peak from a pure sample of crystalline forms of rosiglitazone maleate. The calibration curve may be created in a manner known to those of skill in the art. For example, five or more artificial mixtures of crystalline forms of rosiglitazone maleate, at different amounts, may be prepared. In a non-limiting example, such mixtures may contain, 2%, 5%, 7%, 8%, and 10% of rosiglitazone maleate for each crystalline form. Then, X-ray diffraction patterns are obtained for each artificial mixture using standard X-ray diffraction techniques. Slight variations in peak positions, if any, may be accounted for by adjusting the location of the peak to be measured. The intensities of the selected characteristic peak(s) for each of the artificial mixtures are then plotted against the known weight percentages of the crystalline form. The resulting plot is a calibration curve that allows determination of the amount of the crystalline forms of rosiglitazone maleate in an unknown sample. For the unknown mixture of crystalline and amorphous forms of rosiglitazone maleate, the intensities of the selected characteristic peak(s) in the mixture, relative to an intensity of this peak in a calibration mixture, may be used to determine the percentage of the given crystalline form in the composition, with the remainder determined to be the amorphous material.

In addition to X-ray powder diffraction, amorphous rosiglitazone maleate, or the presence of some amorphous rosiglitazone maleate, can be distinguished from crystalline rosiglitazone maleate, using Raman spectroscopy, solution calorimetry, differential scanning calorimetry, solid state nuclear magnetic resonance spectra (ssNMR) or infra-red spectroscopy. Each of these techniques is well established in the art. Amorphous rosiglitazone maleate can also be identified based on the morphology of the particles seen under an electron microscope. Furthermore, amorphous rosiglitazone maleate is likely to be much more soluble than crystalline rosiglitazone maleate because the former is lack of lattice energy, providing another means of discriminating between the crystalline and amorphous rosiglitazone forms, or detecting an amount of amorphous form within a rosiglitazone maleate preparation. As noted above, the preferred method of differentiating amorphous rosiglitazone from other crystalline and non-crystalline forms of rosiglitazone is X-ray powder diffraction (XPD).

Another method of distinguishing physical forms, such as crystalline and amorphous rosiglitazone, is 13C Solid state NMR spectra (ssNMR) acquired with cross polarization, magic angle spinning and high power proton decoupling. The isotropic chemical shifts (peak positions) measured in solid state NMR spectra are not only a function of the molecule's atomic connectivity, but also of molecular conformation and inter- and intra-molecular interactions. Thus different peak positions may be observed for different physical forms. For amorphous materials, the dispersion of environments often causes substantially broadened spectra.

It will be appreciated that because of the enhanced solubility property of amorphous rosiglitazone maleate, mixtures comprising substantially crystalline or other solid forms of rosiglitazone maleate with amorphous rosiglitazone maleate will, depending on the amount of amorphous product present, may also possess varying degrees of increased solubility. Such mixtures comprising amorphous rosiglitazone maleate can be prepared, for example, by mixing amorphous rosiglitazone maleate prepared according to the present invention with other solid forms of rosiglitazone, such as crystalline form, prepared according to prior art methods. A mixture might also be prepared if the manufacturing process is incomplete, or incorporates steps that allow or cause amorphous product to be formed.

Examples of other solid forms of rosiglitazone maleate include, but are not limited to, crystalline rosiglitazone maleate, and other polymorphs. A detectable amount of amorphous rosiglitazone maleate is an amount that can be detected using conventional techniques, such as FT-IR, Raman spectroscopy, XPD, TMA, DSC and the like.

As noted above, numerous techniques can be employed to detect a particular form of a compound within a mixture. The limits of detection of a particular form in admixture with another form, i.e. crystalline in amorphous or vice versa, is as follows: by XPD it is reported to be approximately 5% according to Hancock and Zografi (J. Pharm. Sci., 86:1-12, 1997) and approximately 2.0% according to Surana and Suryanarayanan (Powder Diffraction, 15:2-6, 2000). The limits of detection by solution calorimetry is reported to be approximately 1% according Hogan and Buckton (International Journal of Pharmaceutics, 207:57-64, 2000). The limits of detection by solid state NMR is reported to be approximately 5-10% according to Saindonet al., (Pharmaceutical Research, 10:197-203,1993). The limits of detection by near infra red spectroscopy is reported to be approximately 2-5% according to Blanco and Villar (Analyst, 125:2311-2314, 2000). The limits of detection by Modulated Differential Scanning Calorimetry (MDSC) is reported to be approximately 6% according to Saklatvala et al., (International Journal of Pharmaceutics, 192: 55-62, 1999). The limits of detection by FTRaman spectroscopy is reported to be approximately 2% according to Taylor and Zografi (Pharm. Res. 15:755-761, 1998).

In another embodiment, the invention provides pharmaceutical compositions comprising the amorphous form of rosiglitazone maleate, which can be formulated with a one or more pharmaceutically acceptable carriers, also known as excipients, which ordinarily lack pharmaceutical activity, but have various useful properties which may, for example, enhance the stability, sterility, bioavailability, and ease of formulation of a pharmaceutical composition. These carriers are pharmaceutically acceptable, meaning that they are not harmful to humans or animals when taken appropriately and are compatible with the other ingredients in a given formulation. The carriers may be solid, semi-solid, or liquid, and may be formulated with the compound in bulk. The resulting mixture may be manufactured in the form of a unit-dose formulation (i.e., a physically discrete unit containing a specific amount of active ingredient) such as a tablet or capsule. The pharmaceutical compositions may include, in addition to a compound of this invention, one or more active pharmaceutical compounds.

The pharmaceutical compositions of the present invention may include one or more additional medicaments in combination with the amorphous form of rosiglitazone maleate.

Generally, the pharmaceutical compositions of the invention may be prepared by uniformly admixing the active ingredient with liquid or solid carriers and then shaping the product into the desired form. The pharmaceutical compositions may be in the form of suspensions, solutions, elixirs, aerosols, or solid dosage forms. Because of their ease of administration, tablets and capsules represent the most advantageous oral dosage unit form, in which case solid pharmaceutical carriers are employed.

A preferred oral solid preparation is a tablet. A tablet may be prepared by direct compression, wet granulation, or molding, of the active ingredient(s) with a carrier and other excipients in a manner known to those skilled in the art. Compressed tablets may be prepared by compressing in a suitable machine the active ingredient in a free-flowing form such as powder or granules, optionally mixed with a binder, lubricant, inert diluent, surface active agent or dispersing agent. Molded tablets may be made on a suitable machine. A mixture of the powdered compound moistened with an inert liquid diluent is suitable in the case of oral solid dosage forms (e.g., powders, capsules, and tablets). If desired, tablets may be coated by standard techniques. The compounds of this invention may be formulated into typical disintegrating tablets, or into controlled or extended release dosage forms.

The pharmaceutical compositions of the invention are contemplated in various formulations suitable for various modes of administration, including but not limited to inhalation, oral, rectal, parenteral (including subcutaneous, intradermal, intramuscular, intravenous), implantable, intravaginal and transdermal administration. The most suitable route of administration in any given case depends on the duration of the subject's condition, the length of treatment desired, the nature and severity of the condition being treated, and the particular formulation that is being used. The formulations may be in bulk or in unit dosage form.

The amount of active ingredient included in a unit dosage form depends on the type of formulation that is formulated. A pharmaceutical composition of the invention will generally include about 0.1% by weight to about 99% by weight of active ingredient, preferably about 1% by weight to 50% by weight for oral administration and about 0.2% by weight to about 20% by weight for parenteral administration.

Formulations suitable for oral administration include capsules (hard and soft), cachets, lozenges, syrups, suppositories, and tablets, each containing a pre-determined amount of the active compound; as a powder or granules; as a solution or a suspension in an aqueous or non-aqueous liquid; or as an oil-in-water or water-in-oil emulsion. Such formulations may be prepared by any suitable method of pharmacy that includes the step of bringing into association the active compound and a suitable carrier or carriers. The amount of active ingredient per unit dosage of solid formulations may be as described in prior art for preparations of rosiglitazone maleate. For liquid oral formulations, a preferable amount is from about 2% by weight to about 20% by weight. Suitable carriers include but are not limited to fillers, binders, lubricants, inert diluents, surface active/dispersing agents, flavorants, antioxidants, bulking and granulating agents, adsorbants, preservatives, emulsifiers, suspending and wetting agents, glidants, disintegrants, buffers and pH-adjusting agents, and colorants. Examples of carriers include celluloses, modified celluloses, cyclodextrins, starches, oils, polyols, sugar alcohols and sugars, and others. For liquid formulations sugar, sugar alcohols, ethanol, water, glycerol, and poyalkylene glycols are particularly suitable, and may also be used in solid formulations. Cyclodextrins may be particularly useful for increasing bioavailability. Formulations for oral administration may optionally include enteric coatings known in the art to prevent degradation of the formulation in the stomach and provide release of the drug in the small intestine. One example of pharmaceutical tablet of the amorphous rosiglitazone maleate may include, as inactive ingredients, hypromellose 2910, lactose monohydrate, magnesium stearate, microcrystalline cellulose, polyethylene glycol 3000, sodium starch glycolate, titanium dioxide, triacetin and 1 or more of synthetic red and yellow iron oxides and talc.

Formulations suitable for buccal or sub-lingual administration include lozenges comprising the active compound in a flavored base, usually sucrose and acacia or tragacanth, although other agents are also suitable, and pastilles comprising the compound in an inert base such as gelatin and glycerin or sucrose and acacia.

Formulations suitable for rectal administration are preferably presented as unit dose suppositories. These may be prepared by admixing the active compound with one or more conventional solid carriers, e.g., cocoa butter, and then shaping the resulting mixture.

The effective amount (i.e., dosage) of active compound for treatment will vary depending on the route of administration, the condition being treated, its severity, and duration, and the state and age of the subject. A skilled physician will monitor the progress of the subject and will adjust the dosage accordingly, depending on whether the goal is to eliminate, alleviate, or prevent a given condition. Generally, the dosage should be considered in proportion to the subject's weight. The daily dose of particular formulations of active compound may be divided among one or several unit dose administrations. For example therapeutic administration about fifteen to thirty minutes before main meals is preferable (i.e. three times daily), although administration of the active compounds may be carried out prophylactically, and may be maintained for prolonged periods of time. One skilled in the art will take such factors into account when determining dosage. Unit dosage of active ingredient may range from about 0.1 mg to about 2 g, preferably from about 1 mg to about 1 g, more preferably from about 1 mg to about 10 mg, even more preferably from about 2 mg to about 8 mg.

In another aspect, the invention also provides methods of for treatment or prophylaxis of diabetes mellitus, conditions associated with diabetes mellitus and complication thereof in a mammal, comprising administering to a patient in need of such treatment an effective amount of a composition comprising the amorphous form of rosiglitazone maleate and one or more pharmaceutically acceptable carrier.

Conditions associated with diabetes include hyperglycaemia and insulin resistance, especially acquired insulin resistance and obesity. Further conditions associated with diabetes include hypertension, cardiovascular disease, especially atherosclerosis, certain eating disorders, in particular the regulation of appetite and food intake in subjects suffering from disorders associated with under-eating ,such as anorexia nervosa, and disorders associated with over-eating, such as obesity and anorexia bulimia. Additional conditions associated with diabetes include polycystic ovarian syndrome and steroid induced insulin resistance.

The complications of conditions associated with diabetes mellitus encompassed herein includes renal disease, especially renal disease associated with the development of Type II diabetes including diabetic nephropathy, glomerulonephritis, glomerular sclerosis, nephrotic syndrome, hypertensive nephrosclerosis and end stage renal disease.

The invention also provides a use of the amorphous rosiglitazone maleate along with one or more additional medicaments. Since rosiglitazone maleate is an insulin sensitivity enhancer, a combination therapy with one or more other medicaments may be particularly desirable. The medicaments that can be used with the amorphous rosiglitazone maleate, for example, include metformin, sulfonylurea, their pharmaceutically acceptable salts, insulin, and combinations thereof. Preferably, the medicament is metformin hydrochloride. The additional medicaments may be admixed with the amorphous rosiglitazone maleate to form a single pharmaceutical dosage form, for example, such as tablets or may be prepared as a separate pharmaceutical dosage form, which can be administered to a patient along with the pharmaceutical dosage form of the amorphous rosiglitazone at the same time or with a time interval depending upon the patients conditions and the additional medicaments being used for the combination therapy. The pharmaceutical dosage forms may include any forms of drug, which are suitable transport the medicaments into body as noted hereinabove. One example of such pharmaceutical dosage form of the combination therapy is a tablet, which may contain rosiglitazone maleate and metformin hydrochloride equivalent to: 1 mg rosiglitazone with 500 mg metformin hydrochloride, 2 mg rosiglitazone maleate with 500 mg metformin hydrochloride, or 4 mg rosiglitazone maleate with 500 mg metformin hydrochloride in addition to inactive ingredients such as hypromellose, lactose monohydrate, magnesium stearate, microcrystalline cellulose, polyethylene glycol 400, povidone 29-32, sodium starch glycolate, titanium dioxide and one or more of red and yellow iron oxides.

The invention is further described by reference to the following examples which set forth in detail the preparation of compounds and compositions of the present invention, as well as their utility. It will be apparent to those skilled in the art, that many modifications, both to materials, and methods, may be practiced without departing from the purpose and interest of this invention. The examples that follow are not intended to limit the scope of the invention as described hereinabove or as claimed below.

### REFERENCE EXAMPLE 1

Preparation of Rosiglitazone Maleate

5-[4-[2-(N-Methyl-N-(2-pyridyl)amino)ethoxy]benzyl]thiazolidine-2,4-dione (470 g) and maleic acid (137 g) were dissolved in ethanol (41 L) at boiling. The hot solution was filtered via diatomaceous earth and was then allowed to cool slowly with gentle agitation. After leaving in a refrigerator at 0-5° C. for several hours, the maleate salt was filtered off, washed with ethanol and dried in vacuum at 50° C. to give 446 g (73%) of product.

Process for the Preparation of Amorphous Form of Rosiglitazone Maleate.

### EXAMPLE 1

Dissolved was 20 grams of Rosiglitazone Maleate in 600 mL of methanol with stirring while the reaction mixture was heated to 60 to 70° C. The solvent was completely distilled off under reduced pressure at 60 to 70° C. Via scratching, the obtained solid was separated from the reactor to afford the desired amorphous form of Rosiglitazone Maleate (18.5 grams, 92.5% of yield).

### EXAMPLE 2

Dissolved was 20 grams of Rosiglitazone Maleate in 600 ml of acetonitrile with stirring while the reaction mixture was heated to 60 to 70° C. The solvent was completely distilled off under reduced pressure at 60 to 70° C. By scratching, the obtained solid was separated from the reactor to afford the desired amorphous form of Rosiglitazone Maleate (18.5 grams, 92.5% of yield).

We claim

1. A compound which is an amorphous form of rosiglitazone maleate.

2. The compound of claim 1 having substantially the same X-ray diffraction pattern as shown in FIG. 1.

3. A composition comprising rosiglitazone maleate as a solid, wherein at least 80% by weight of said solid rosiglitazone maleate is in an amorphous form.

4. The composition of claim 3, wherein at least 95% of said solid rosiglitazone maleate is in said amorphous form.

5. The composition of claim 3, wherein at least 99% of said solid rosiglitazone maleate is in said amorphous form.

6. The composition of claim 3, which is substantially free of crystalline forms of rosiglitazone maleate.

7. A process for making an amorphous form of rosiglitazone maleate, said process comprising:
a. providing rosiglitazone maleate as a solution in a solvent selected from the group consisting of a nitrile solvent, an alcoholic solvent, and/or a mixture thereof;
b. removing solvent at an elevated temperature to form a solid residue; and
c. isolating said solid residue, which is said amorphous form of rosiglitazone maleate.

8. The process of claim 7, wherein said solvent is selected from the group consisting of acetonitrile, propionitrile, methanol, ethanol, isopropyalcohol, n-butyl alcohol, t-butyl alcohol and mixtures thereof.

9. The process of claim 7, wherein said solvent is acetonitrile.

10. The process of claim 7, wherein said solvent is methanol.

11. A compound which the amorphous form of rosiglitazone produced by the process of claim 7.

12. A pharmaceutical composition comprising the compound of claim 1 and one or more pharmaceutically acceptable carriers.

13. The pharmaceutical composition of claim 12, further comprising at least one additional active ingredient.

14. The pharmaceutical composition of claim 13, wherein said additional active ingredient is selected from the group consisting of metformin, sulfonyl urea, insulin, and pharmaceutically acceptable thereof.

15. The pharmaceutical composition of claim 13, wherein said additional active ingredient is metformin hydrochloride.

16. A method for the treatment or prophylaxis of diabetes mellitus, conditions associated with diabetes mellitus and complication thereof which comprises administering an effective amount of the compound of claim 1 to a mammal in need of said treatment or prophylaxis.

17. The method of claim 16, wherein said method further comprises administering metformin hydrochloride in combination with the compound of claim 1.

18. The method of claim 16, wherein said mammal is a human.
